(19) United States
(12) Patent Application Publication
(10) Pub. No.: US 2009/0247754 A1
(43) Pub. Date: Oct. 1, 2009

(54) METHOD OF PREPARING HUPERZINE A AND DERIVATIVES THEREOF

(75) Inventors: Gail Underiner, Malvern, PA (US); Frank Gibson, Pennington, NJ (US); Linli He, West Chester, PA (US); Harihara Subramanian Meera, Chennai (IN); Jesudoss Mercy Gnanadeepam, Chennai (IN); Ramanathan Saiganesh, Chennai (IN); Stephen R. Tudhope, County Durham (GB); Manouchehr Azadi-Ardakani, Newcastle upon Tyne (GB)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: DEBIOPHARM S.A.

(21) Appl. No.: 12/411,111

(22) Filed: Mar. 25, 2009

Related U.S. Application Data
(60) Provisional application No. 61/039,233, filed on Mar. 25, 2008.

(51) Int. Cl. C07D 221/22; C07D 215/00

(52) U.S. Cl. 546/97; 546/153

(57) ABSTRACT

The present invention is related to the synthesis of huperzine A. The synthesis includes a variety of process steps that increase productivity, reduce safety concerns, and allow for increasing production of compounds of desired optical isomer. The inventive methods may encompass a single improved reaction step that may be incorporated into a known reaction process for synthesizing huperzine A or a derivative thereof to improve the overall reaction. The inventive methods also encompass complete synthesis methods for preparing huperzine A or a derivative thereof.

### CROSS-REFERENCE TO RELATED APPLICATIONS

The present application claims priority to U.S. Provisional Patent Application No. 61/039,233, filed Mar. 25, 2008, which is incorporated herein by reference in its entirety.

### FIELD OF THE INVENTION

The present invention is directed to methods for the synthesis of huperzine A, as well as analogs and derivatives thereof.

### BACKGROUND

Huperzine A is a plant alkaloid derived from the Chinese club moss plant, Huperzia serrata, which is a member of the Lycopodium species. Huperzia serrata has been used for centuries in Chinese medicine for the treatment of many conditions, including fevers, blood disorders, and inflammation.

In addition to these historical uses, huperzine A has more recently been found to exhibit useful neuroprotective effects. In particular, clinical trials have shown huperzine A to have acetylcholinesterase activity making it useful for increasing acetylcholine levels in the brain following administration. It also increases norepinephrine and dopamine levels, but not serotonin levels. In light of its neuroprotective effects, particularly its ability to affect acetylcholine levels, huperzine A is currently being investigated as a possible treatment for diseases characterized by neurodegeneration, including myasthenia gravis, senile memory loss, and Alzheimer's disease.

Alzheimer's disease is a neurodegenerative disorder associated with neuritic plaques that affect the cerebral cortex, amygdala, and hippocampus. Alzheimer's disease is also characterized by neurotransmission damage in the brain, and one of the major functional deficits in Alzheimer's disease is a hypofunction of cholinergic neurons. This leads to the cholinergic hypothesis of Alzheimer's disease and the rationale for strategies to increase acetylcholine in the brains of Alzheimer's disease patients.

Known drugs used for the treatment of Alzheimer's disease, such as tacrine, galantamine, and donepezil, are also acetylcholinesterase inhibitors. Clinical trials in China have shown that huperzine A is comparably effective to the known treatments and may even have increased safety in light of fewer side effects. Because of these useful activities of huperzine A, development of methods of synthesizing the compound is highly desirable. Likewise, it is desirable to determine methods of synthesizing analogs and derivatives of huperzine A. Accordingly, it would be useful to have further methods for synthesizing huperzine A for use itself as a pharmaceutical agent, but also for use as a starting material for the synthesis of analogs and derivatives of huperzine A.

### SUMMARY OF THE INVENTION

The present invention provides methods of preparing huperzine A. The compounds prepared according to the methods of the invention can be used themselves as pharmaceutical agents or as starting materials for further synthetic methods. Accordingly, the invention also provides methods of preparing analogs and derivatives of huperzine A. Still further, the invention provides methods of preparing compounds having pharmacological activity, wherein huperzine A is prepared as an intermediate in the process.

In one embodiment, the invention provides a method of preparing huperzine A, the method comprising the following steps:

A) reacting the compound of Formula (1) with methyl propiolate to form the compound of Formula (2)

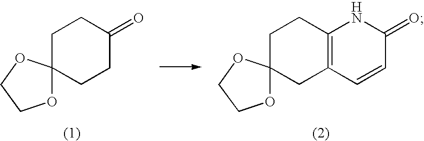

B) methylating the compound of Formula (2) to form the compound of Formula (3)

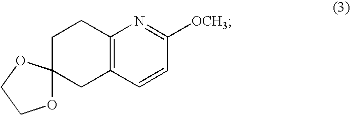

C) performing acid hydrolysis on the compound of Formula (3) to form the compound of Formula (4)

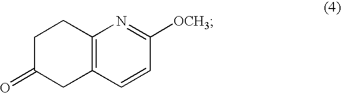

D) performing carboxymethylation on the compound of Formula (4) to form the compound of Formula (5)

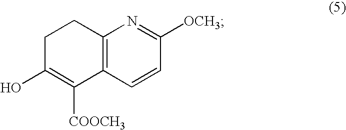

E) performing annulation on the compound of Formula (5) to form the compound of Formula (6)

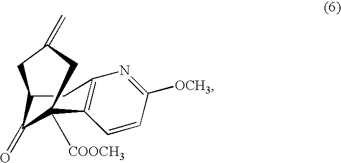

and performing isomerization on the compound of Formula (6) to form the compound of Formula (7)

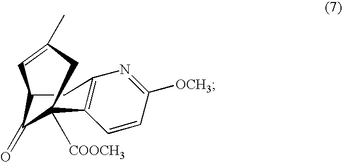

F) performing Wittig coupling on the compound of Formula (7) to form the compound of Formula (8)

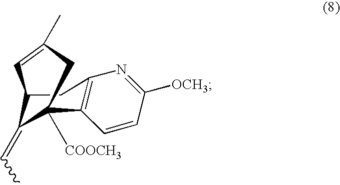

G) performing isomerization on the compound of Formula (8) to form the compound of Formula (9)

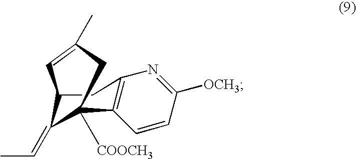

H) performing base hydrolysis on the compound of Formula (9) to form the compound of Formula (10)

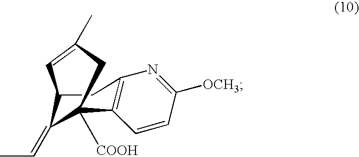

I) performing a Curtius rearrangement on the compound of Formula (10) to form the compound of Formula (11)

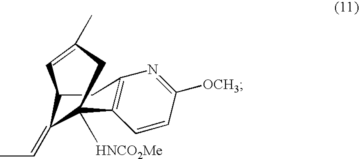

and

J) performing carbamate hydrolysis and cleavage of the methyl ester of Formula (11) to form huperzine A according to Formula (12)

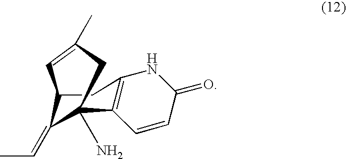

In a specific embodiment, the present invention provides a method of preparing huperzine A, the method comprising the step of converting an intermediate compound of the Formula (3)

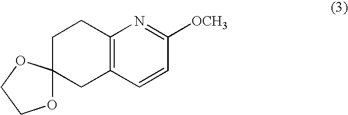

to an intermediate compound of the Formula (4)

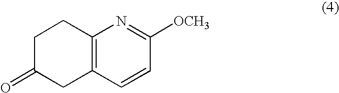

by acid hydrolysis, wherein the acid comprises aqueous phosphoric acid. What could not have been foreseen prior to the present invention is that the specific use of phosphoric acid for carrying out the hydrolysis leads to a surprising and significant reduction in the amount of time required to complete the hydrolysis and convert the compound of Formula (3) into the compound of Formula (4). Whereas a typical method of hydrolyzing a compound according to Formula (3) requires a time of about 16 hours to reach completion, the method of the present invention, in certain embodiments, facilitates completion of the hydrolysis in a time of less than about 3 hours. In specific embodiments, such completion is evidenced by the reaction mixture comprising less than about 2% by weight of the original amount of the compound of Formula (3) introduced into the reaction. This specific reaction step could be used in any known method of preparing huperzine A to provide an improved process according to the present invention.

In another specific embodiment, the method of the invention comprises the step of converting the compound of Formula (4) to an intermediate compound according to Formula (5)

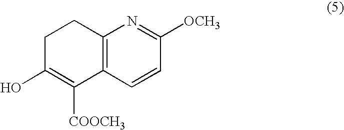

by contacting the compound of Formula (4) with sodium hydride in dimethyl carbonate. Preferably, the dimethyl carbonate is present in an amount suitable to function as a reagent and a solvent. In specific embodiments, the method step is carried out in the express absence of tetrahydrofuran as a solvent. Preferably, the compound of Formula (5) prepared according to this method exhibits a purity, when measured by HPLC, of at least about 90%. This specific reaction step could be used in any known method of preparing huperzine A to provide an improved process according to the present invention.

In yet another specific embodiment, the inventive method comprises converting a compound of the Formula (5) into a compound of the Formula (6)

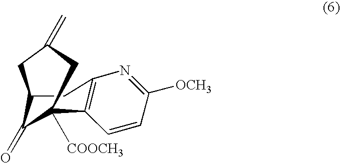

by performing an annulation reaction using acetone as the reaction solvent and converting the compound of Formula (6) into a compound of Formula (7)

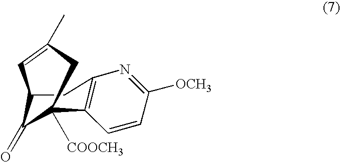

by performing an isomerization reaction using ethylene dichloride as the reaction solvent. In certain embodiments, the annulation can further comprise increasing optical purity of the compound of Formula (6) by carrying out a recrystallization. Surprisingly, according to the invention, the increase in optical purity can be achieved by carrying out only a single recrystallization of the compound of Formula (6) by using isopropyl alcohol as the recrystallization solvent. Such recrystallization can result in an optical purity of at least about 90%.

In other embodiments, the isomerization reaction can be carried out at beneficial reaction conditions. For example, the isomerization reaction can be carried out at a temperature of less than 30° C. and can be completed in a time of less than four hours. This specific reaction step could be used in any known method of preparing huperzine A to provide an improved process according to the present invention.

In other embodiments, the inventive method comprises performing isomerization on the compound of Formula (8)

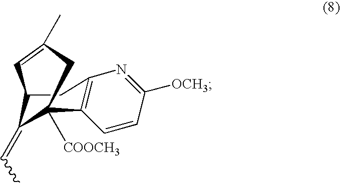

to form the compound of Formula (9)

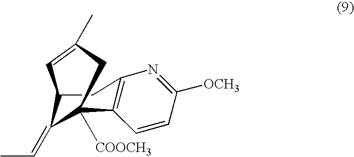

by reacting the compound of Formula (8) with thiophenol. Preferably, the thiophenol is activated with an activating material. In a specific embodiment, the activating material is zinc.

In further embodiments, the disclosed methods can be used individually, or in combinations, in an overall method for preparing huperzine A. Moreover, the huperzine A can be subjected to further process steps to prepare a variety of analogs and derivatives of huperzine A.

### DETAILED DESCRIPTION

The present invention now will be described more fully hereinafter with reference to preferred embodiments. These embodiments are provided so that this disclosure will be thorough and complete, and will fully convey the scope of the invention to those skilled in the art. Indeed, the invention may be embodied in many different forms and should not be construed as limited to the embodiments set forth herein; rather, these embodiments are provided so that this disclosure will satisfy applicable legal requirements. Like numbers refer to like elements throughout. As used in the specification, and in the appended claims, the singular forms “a”, “an”, “the”, include plural referents unless the context clearly dictates otherwise.

Huperzine A is a compound having the structure illustrated in Formula (12) and is also known as HUP, hup A, selagine and, in Chinese medicine, Chien Tseng Ta and shuangyiping.

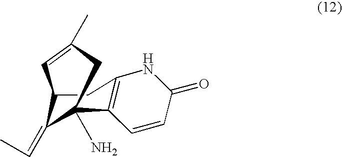

Huperzine A is a chiral molecule that can exist as the L- or (−) isomer, the D- or (+) isomer, or as a racemic mixture, or (+/−)-huperzine A. While natural huperzine A exists in the (−) form, synthetic huperzine A is the racemic mixture. Since the in vitro activity of pure (−)-huperzine A is approximately three times greater than racemic or (+/−)-huperzine A, known synthetic methods of preparing huperzine A are intrinsically inferior to simply isolating natural huperzine A because of the much greater potency of the naturally-derived compound.

Because synthesized huperzine A is formed as the racemic mixture, known synthetic methods result in a huperzine A product that either exhibits reduced activity (because of the content of (+) huperzine A) or reduced yield (because of the need to remove the undesired isomer). Techniques for increasing optical purity in known synthetic methods typically suffer from an inability to achieve a sufficiently high degree of enantiomeric excess of the desired (−)-huperzine A to avoid a significant loss of product. In specific embodiments, as more fully described below, the present invention particularly overcomes this limitation.

The methods of the present invention also provide various further improvements over known synthetic methods for preparing huperzine A. In particular, the inventive method provides a number of steps that reduce overall time of synthesis, increase yield of the desired isomer, and reduce or eliminate health and safety issues common to known synthetic methods. Each of the specific synthesis steps described herein individually could be incorporated into known methods of preparing huperzine A as each specific synthesis step individually provides improvements over the known methods. Thus, the invention encompasses methods characterized by incorporating a single specific synthesis step as described herein. Of course, combinations of the various specific synthesis steps described herein can provide improved results to an even greater degree than the use of a single specific synthesis step in a known method. While the invention is described below in terms of the individual synthesis steps that can provide improvements to an overall synthesis method, it is understood that the invention encompasses any entire synthesis method that incorporates one or more of the specifically described synthesis steps.

In one aspect, the present invention provides a method for the synthesis of huperzine A. The overall method of the invention is characterized by various process steps that each individually are improvements over known synthetic methods and facilitate the production of synthetic huperzine A that can be used directly in pharmaceutical formulations and method or can be used in the preparation of analogs and derivatives of huperzine A.

In one embodiment, the method of the invention includes an acid hydrolysis step wherein an intermediate ketal compound according to Formula (3) is converted to an intermediate ketone compound according to Formula (4).

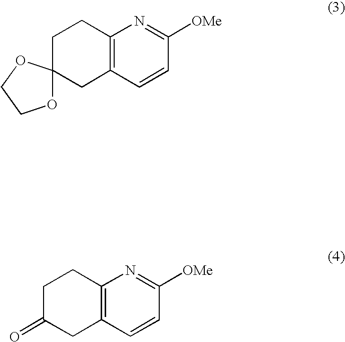

Acid hydrolysis can be carried out using a variety of reactants. For example, one method comprises the use of dilute hydrochloric acid in acetone; however, the use of such reactants raises concerns about toxic side products and requires long reaction times and a complicated workup that requires the use of chromatography.

Other reactants may also be used, such as trifluoro acetic acid, sulfuric acid, nitric acid, and acetic acid. While such acids are useful for effecting hydrolysis, in a preferred embodiment, acid hydrolysis is carried out using aqueous phosphoric acid. What could not have been foreseen is that phosphoric acid provides for a clean, rapid hydrolysis of the ketal to the ketone that provides distinct advantages over not only hydrochloric acid, but also other acids. Hydrolysis using HCl requires an extended reaction time on the order of greater than 16 hours. Surprisingly, the time for hydrolysis using phosphoric acid is reduced by as much as, or even greater than, a full order of magnitude. In light of these advantages, the use of phosphoric acid for carrying out hydrolysis is well beyond simple arbitrary choice from a number of possible acids. Rather, phosphoric acid improves the overall synthesis in a surprising manner.

Hydrolysis according to the invention using phosphoric acid as opposed to a different acid makes it possible to achieve complete conversion of the ketal to the ketone in a time of less than about 5 hours. As used herein, the conversion of ketal to ketone is considered complete when the reaction mixture comprises less than about 5% by weight of the original amount of the starting material—i.e., the ketal compound (Formula 3) that was introduced into the reaction. In further embodiments, the reaction is considered complete when the reaction mixture comprises less than about 4% by weight, less than about 3% by weight, less than about 2% by weight, less than about 1% by weight, or less than about 0.5% by weight of the original amount of the ketal compound introduced into the reaction.

In specific embodiments, hydrolysis according to the invention using phosphoric acid is useful for facilitating completion of the hydrolysis reaction in a time of less than about 4 hours. Preferably, hydrolysis is complete in a time of less than about 3 hours. In one embodiment, hydrolysis is complete in a time of about 1-2 hours.

In certain embodiments, time to achieving complete reaction can be further defined by certain reaction parameters. For example, time to complete reaction can be defined in terms of the reaction temperature. In some embodiments, hydrolysis according to the invention using phosphoric acid is carried out at a reaction temperature of about 60° C. to about 95° C., preferably about 65° C. to about 90° C., more preferably about 70° C. to about 85° C., still more preferably about 75° C. to about 80° C.

Time to complete reaction can also be defined in terms of the volume of phosphoric acid and the volume of water used in the reaction in relation to the ketal starting material. In specific embodiments, the hydrolysis reaction is carried out such that the volume to volume ratio of phosphoric acid to ketal starting material is from about 4:1 to about 1:4, about 3:1 to about 1:3, about 2:1 to about 1:2, or about 1.5:1 to about 1:1.5. In specific embodiments the ratio of phosphoric acid to ketal starting material is about 1:1. The volume to volume ratio of water to ketal starting material is from about 12:1 to about 6:1, about 11:1 to about 7:1, or about 10:1 to about 8:1. In specific embodiments, the ratio of water to ketal starting material is about 9:1. In light of the above, time to complete reaction can also be defined in terms of the volume of water to the volume of phosphoric acid used in the hydrolysis reaction. In certain embodiments, the volume to volume ratio of water to phosphoric acid is about 12:1 to about 6:1, about 11:1 to about 7:1, or about 10:1 to about 8:1. In specific embodiments, the ratio of water to phosphoric acid is about 9:1.

Time to complete reaction can be evaluated using any methods capable of determining the content of ketal starting material present in a sample of the reaction mixture. In one embodiment, evaluating time to complete reaction can be carried out by intermittently or continuously testing a sample of the reaction mixture using high pressure liquid chromatography (HPLC) analysis.

According to one embodiment, the invention provides a method comprising the step of converting a compound of Formula (3) to a compound of Formula (4) by hydrolysis using phosphoric acid, wherein, after heating the compound of Formula (3) in a mixture with phosphoric acid and water at a temperature of about 70° C. to about 85° C. for a time of less than 5 hours (preferably less than 4 hours or less than 3 hours), the compound of Formula (3) is completely converted to the compound of Formula (4) such that less than about 2% by weight of the compound of Formula (3) (preferably less than about 1% by weight) remains. Similarly, the conversion can be carried out to a point of completion as further defined above in an amount of time as further described above.

In one embodiment, as illustrated in Example 1 below, the method of the invention can be carried out such that the compound of Formula (3) is converted to the compound of Formula (4), with <1% of the starting material remaining, in a time of only about 3 hours. In such embodiment, the compound of Formula (3) was combined with water and phosphoric acid (88%), dissolved, and heated to the reaction temperature of 75-80° C., where the reaction was maintained for the noted time.

Varying concentrations of phosphoric acid can be used while still achieving the surprising and significant decrease in the time to complete hydrolysis. Preferably, the phosphoric acid has a concentration of about 1M to about 6M.

In another embodiment, the method of the invention includes a methoxycarbonylation step for converting the compound of Formula (4) to an intermediate compound according to Formula (5) by contacting the compound of Formula (4) with sodium hydride in dimethyl carbonate.

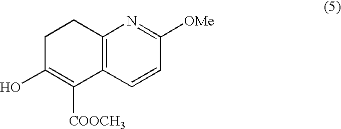

Sodium hydride is particularly useful in the inventive method because of the problems associated with the use of other hydrides, such as potassium hydride. For example, when using potassium hydride, chromatography must be used to obtain a solid product. Moreover, potassium hydride has associated safety concerns.

Sodium hydride is also useful in light of its improved results over other bases that may be used as an alternative to potassium hydride. For example, sodium-, lithium-, or potassium-hexamethyldisilazane (NaHMDS), (LiHMDS), or (KHMDS), as well as Li⁺, Na⁺, and K⁺ t-butoxides may all be used for carrying out the methoxycarbonylation, but all of these alternative bases facilitate only a small conversion to the compound of Formula (5). Moreover, all of these alternative bases result in the formation of multiple impurities. Surprisingly, however, sodium hydride provides for excellent yield of the desired β-ketoester (Formula 5) while maintaining good purity. Furthermore, the use of sodium hydride is advantageous over the use of potassium hydride because of reduced safety concerns.

In specific embodiments of the invention, the method of preparing the methyl ester of Formula (5) is particularly characterized by the improved purity of the synthesized compound. For example, in one method of preparing the methyl ester of Formula (5), 1 equivalent of the ketone (Formula 4) can be combined with 2 equivalents of sodium hydride and 13.5 equivalents of dimethyl carbonate in 50 volumes of THF. The reaction is carried out at a temperature of about 0° C., and the resultant white solid ester (Formula 5) has a purity of about 50-60%.

Surprisingly, however, purity can be greatly increased by eliminating the addition of a dedicated solvent (i.e., material introduced as a solvent alone that is not also a reagent in the reaction). In one particular embodiment, the method is specifically carried out in the absence of THF as a solvent. Preferably, the amount of dimethyl carbonate is increased to account for the eliminated additional solvent. In other words, the dimethyl carbonate is present in an amount suitable to function as a reagent and a solvent.

In one embodiment, 1 equivalent of the ketone (Formula 4) can be combined with 1.2 equivalents of sodium hydride and about 15-30 volumes of dimethyl carbonate. The reaction is carried out without the addition of any further solvent. Preferably, the reaction temperature is raised, such as to a temperature of about 90° C. Under these conditions, the resultant white solid ester (Formula 5) has a purity of about 90-98%. Increased purity particularly can be obtained by recrystallization using a mixture of hexane and ethyl acetate.

In light of the above, in one embodiment, the present invention provides a method for synthesizing an intermediate compound according to Formula (5) having a high degree of purity. Preferably, the synthesized compound has a purity, when measured by HPLC, of at least about 90%, at least about 92%, at least about 95%, at least about 97%, or at least about 98%. Particularly, the method comprises reacting a compound according to Formula (4) with sodium hydride and dimethyl carbonate in the express absence of a dedicated solvent. In a preferred embodiment, the reaction is carried out in the express absence of THF as a dedicated solvent. In particular embodiments, the reaction is carried out using dimethyl carbonate as a reactant and as a solvent. In a further preferred embodiment, the reaction is carried out at a temperature of about 90° C.

As noted above, the invention expressly encompasses methods comprising a methoxycarbonylation step using sodium hydride as a reactant. The invention, however, also encompasses methods that use potassium hydride as a reactant in the methoxycarbonylation step. If sodium hydride is not used in the methoxycarbonylation step, it is preferred that the overall synthesis method incorporate one or more of the further specific reaction steps described herein that are useful to improve the overall method for synthesizing huperzine A.

In yet another embodiment, the present invention provides improvements in a method for preparing huperzine A relating to a step comprising annulation and acid isomerization. In particular, the method of the invention allows for the use of milder reaction conditions during annulation, which alone is beneficial. However, the improvement also allows for increasing optical purity, which increase can arise from improvements to the annulation step, as well as later recrystallization steps.

During annulation, the compound according to Formula (5) is reacted with a chiral ligand, allyl palladiumchloride dimer, and 2-methylene-1,3-propanediol diacetate using acetone as a solvent. It was found according to the present invention that the use of acetone as a solvent provided surprising and unexpected results in comparison to the use of other solvents, such as toluene. For example, the use of acetone allows for the reaction to be carried out at a temperature that is close to ambient (i.e., around 5-20° C.). It is surprising that, even though reaction temperature is changed, the time to complete reaction is greatly reduced. In specific embodiments, the time to completion of the annulation reaction is less than 5 hours, less than 4 hours, less than 3 hours, or less than 2 hours. Typically, annulation reactions in the preparation of huperzine A take on the order of 15 hours to complete. The use of acetone as the solvent also simplifies solvent removal. For example, the acetone can simply be distilled off (e.g., at a temperature of about 40-45° C.).

Surprisingly, the use of acetone as the solvent also improves optical purity of the annulation product and simplifies the purification process. According to the present invention, the crude annulation product—i.e., the compound according to Formula (6)—can be purified by a single recrystallization using isopropyl alcohol. In specific embodiments, a single recrystallization using IPA can result in the compound according to Formula (6) having an optical purity of at least about 95%, at least about 96%, at least about 97%, at least about 98%, or at least about 99%.

Accordingly, in certain embodiments, the method of the invention includes an annulation step for preparing a compound according to Formula (6),

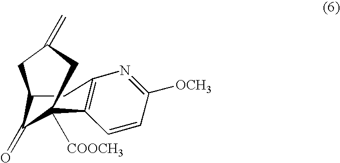

wherein the annulation is carried out at a temperature of less than 30° C., preferably about 5-20° C., and wherein the reaction is carried out using acetone as the reaction solvent. The annulation can further comprise a purification step, wherein purification comprises a single recrystallization using isopropyl alcohol as the recrystallization solvent.

In the isomerization portion of the method, the compound according to Formula (6) is converted into the compound according to Formula (7).

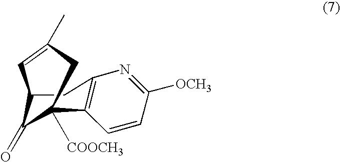

In known methods, annulation and isomerization results in production of a crude intermediate product of unacceptable enantiomeric excess. The present invention, however, overcomes this limitation.

Enantiomeric excess is understood to exist where a chemical substance comprises two enantiomers of the same compound and one enantiomer is present in a greater amount than the other enantiomer. Unlike racemic mixtures, these mixtures will show a net optical rotation. With knowledge of the specific rotation of the mixture and the specific rotation of the pure enantiomer, the enantiomeric excess (abbreviated “ee”) can be determined by known methods. Direct determination of the quantities of each enantiomer present in the mixture is possible with NMR spectroscopy and chiral column chromatography.

As previously noted, the in vitro activity of pure (−)-huperzine A is approximately three times greater than racemic or (+/−)-huperzine A. Accordingly, it would be highly useful to have a method for preparing huperzine A wherein the process itself includes a step for increasing optical purity. This increases the intrinsic activity of the end product and reduces the cost and effort needed to otherwise improve optical activity. Specifically, the inventive method avoids the need to perform costly separation methods to isolate the desired isomer from an enantiomeric mixture and also eliminates waste product in the form of the undesired enantiomer.

The isomerization step comprises reacting the compound of Formula (6) with trifluoromethanesulphonic acid using ethylene dichloride as the solvent. Surprisingly, the use of this solvent rather than another solvent, particularly 1,4-dioxane, improves the overall process by reducing reaction temperature and reaction time. For example, when using ethylene dichloride as the reaction solvent according to the invention, the isomerization step can be carried out at a temperature of less than 30° C., less than 25° C., or around 20° C. In some embodiments, isomerization reaction temperature is about 5° C. to about 30° C., about 5° C. to about 25° C., about 10° C. to about 25° C., or about 15° C. to about 25° C. Reaction using 1,4-dioxane as the solvent typically is carried out at a temperature of around 80° C. Moreover, despite the drastic reduction in reaction temperature, the isomerization reaction according to the invention can be completed in a time of less than about 3 hours, less than about 2 hours, or about 1 hour. Reaction using 1,4-dioxane as the solvent typically requires a reaction time of about 15 hours.

The methods described herein, separately or together, beneficially can be incorporated into an overall process for the synthesis of huperzine A. Thus, the present invention provides for multiple methods of preparing huperzine, the multiple methods individually incorporating a single improved reactions step described herein or incorporating one or more combinations of the improved reaction steps described herein. One route to the synthesis of huperzine A according to the present invention is illustrated below in Reaction Scheme I.

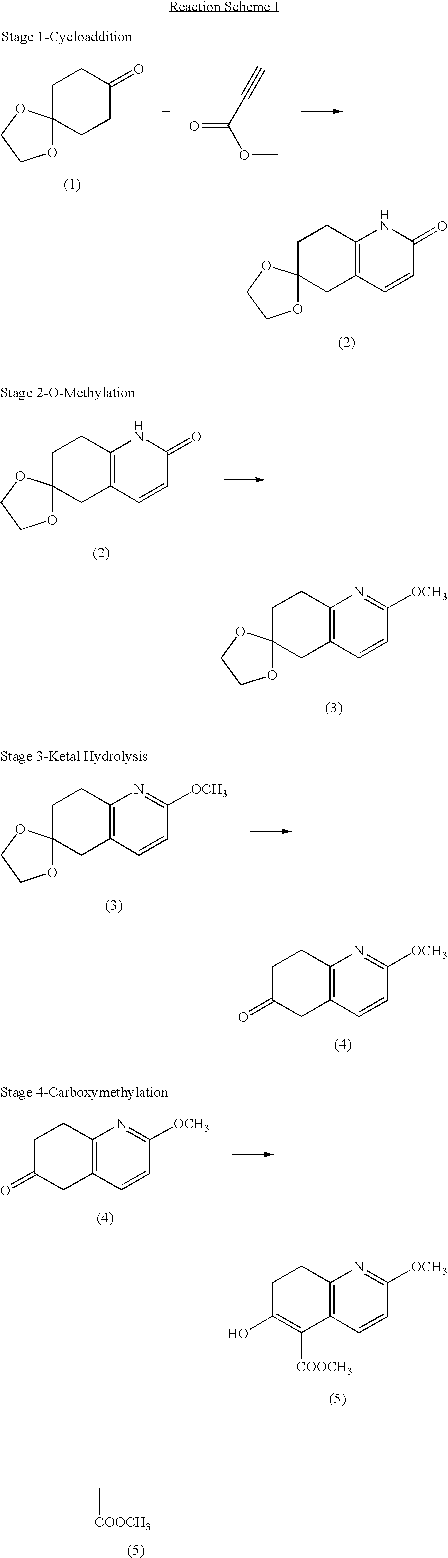

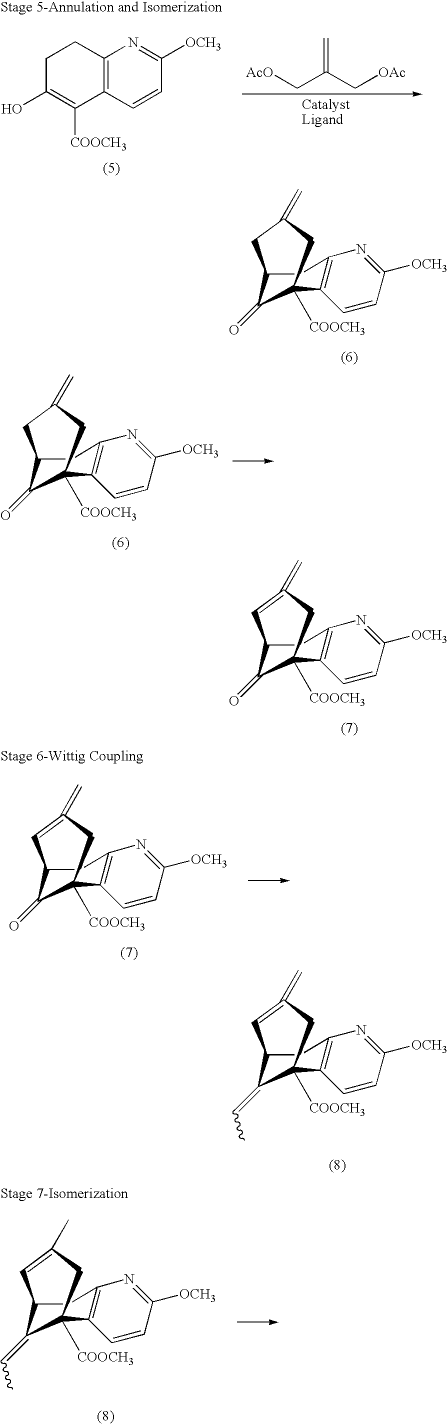

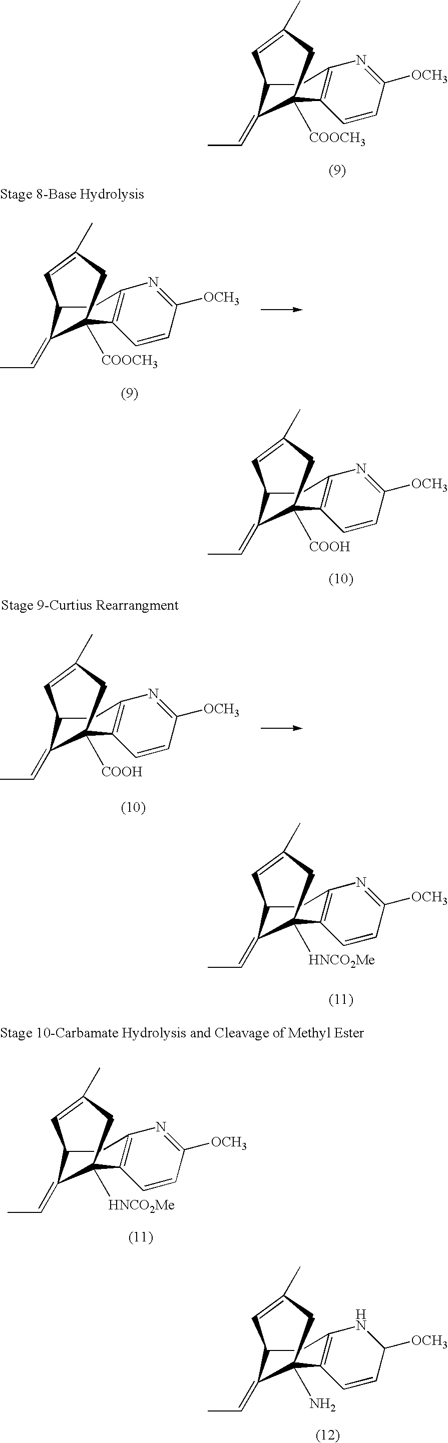

In certain embodiments, the general reaction scheme shown above can be carried out using a variety of specific reactants, solvents, and reaction conditions. In one embodiment, the reaction can be carried out according to the following methods.

In Stage 1, 1, 4-Cyclohexanedione monoethylene ketal (Formula 1) can be reacted with methyl propiolate to prepare the pyridone 1′,5′,7′,8′-tetrahydro-spiro[1,3-dioxolane-2,6′(2′H)-quinolin]-2′-one (Formula 2). The ketal can be placed in a reactor with a suitable solvent, such as isopropyl alcohol, and ammonia in methanol. After addition of the methyl propiolate, the reaction can be heated for a time sufficient for precipitate to form. After precipitate has formed, the reaction slurry can be filtered and the pyridone of Formula 2 can be filtered and collected.

The specific reaction conditions can affect the yield of the reaction. For example, in one embodiment, 1 equivalent of the ketal can be reacted with 1.2 equivalents of methyl propiolate in 20 volumes of isopropyl alcohol and 6 volumes of 7N methanolic ammonia. After heating to a temperature of about 90° C. for a time of about 1.5 hours, about an 18% yield of the pyridone can be obtained.

In one embodiment, 1 equivalent of the ketal can be reacted with 2.1 equivalents of methyl propiolate in 16 volumes of isopropyl alcohol and 10.5 volumes of 7-8N methanolic ammonia. After heating to a temperature of about 135-140° C. for a time of about 9 hours, a pyridone yield of about 42-44% can be obtained.

In Stage 2, the compound of Formula 2 can be methylated to form the tetrahydroquinoline of Formula 3. The pyridone can be charged into a vessel with a suitable phase transfer catalyst, such as benzyl triethylammonium chloride) and with a suitable solvent, such as dichloromethane (DCM). To this mixture a silver-containing base, such as silver carbonate, can be added with a methyl salt, such as dimethyl sulfate or iodomethane. After reaction completion, the silver salts can be removed, such as by filtration, and the O-methylated product can be used in the next stage. In certain embodiments, the reaction product can comprise a two-phase solution that can be used directly in the next synthesis step.

Stage 3 hydrolysis can be carried out by adding a suitable acid to the solution from the previous step including the compound of Formula 3. In preferred embodiments, as described above, the acid comprises aqueous phosphoric acid. Additional solvent (e.g., water) can be added, if needed. The mixture can be heated to allow any solvent to distill away. Mixture pH can be adjusted, if necessary, to maintain a pH that is only slightly acidic (e.g., about 6.5), such as by adding NaOH. A suitable organic solvent, such as ethyl acetate, can be used to extract the ketone of Formula 4 (2-methoxy-7,8-dihydro-5H-quinolin-6-one). The oil can be used directly in the next reaction step or stored under nitrogen until reaction proceeds.

Alternately, the ketone can be directly isolated from the crystallized compound of Formula 3. For example, the ketal can be dissolved in the phosphoric acid and pH adjusted with base. Ethyl acetate extraction can be carried out and a precipitate of the ketone formed by addition of heptane.

Carboxymethylation of the ketone of Formula 4 can be carried out in Stage 4, wherein the deprotected ketone of Formula 4 can be combined with a base, such as sodium hydride, and a solvent, such as dimethyl carbonate. A suitable acid, such as HCl, can be added to quench the reaction by lowering the pH, such as to about pH 3. From the aqueous layer is extracted 6-hydroxy-2-methoxy-7,8-dihydroquinoline-5-carboxylic acid methyl ester (Formula 5) as a white solid. In alternate embodiments, sodium hydride could be substituted with other suitable bases, such as potassium hydride, so long as the reaction also includes one or more other reaction steps described herein as providing improved reaction results.

Stage 5 involves two reaction steps and comprises annulation to form 5-methoxy-11-methylene-13-oxo-6-aza-tricyclo[7.3.1.0]trideca-2(7),3,5-triene-1-carboxylic acid methyl ester (Formula 6) followed by isomerization to form 5-methoxy-11-methyl-13-oxo-6-aza-tricyclo[7.3.1.0]trideca-2(7),3,5,10-tetraene-1-carboxylic acid methyl ester (Formula 7). Annulation can be carried out by reacting the compound of Formula 5 with allylic diacetate over a suitable catalyst and in the presence of a chiral ligand. A variety of ligands can be used, such as ferrocenyl ligands and ligands typically known as Terashima ligands or Hayashi ligands. One specific example of a chiral ligand useful according to the invention is provided below in Formula (13).

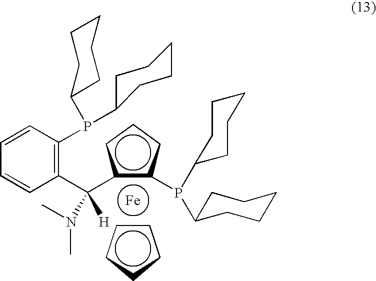

In a preferred embodiment, the catalyst is an allylpalladium chloride dimer. The compound of Formula 6 can be isolated and used directly in the next stage without any further purification.

The crude compound can be charged into a reactor with trifluoromethanesulfonic acid and a solvent, such as anhydrous 1,4-dioxane or, preferably, ethylene dichloride. The resulting reaction mixture can be heated to reaction completion and the obtained residue can be extracted. The extracted compound of Formula 7 can be recrystallized to increase optical purity. Such recrystallization is described above.

The optically purified compound of Formula 7 can be reacted in Stage 6 to undergo Wittig coupling. Specifically, a reactant mixture of a phosphonium bromide, anhydrous THF, and an organo lithium can be prepared and combined with the β-ketoester of Formula 7. The achieved Wittig product (Formula 8) is a mixture of Z- and E-isomers, typically in a low ratio, such as about 3 to 1 E:Z.

In Stage 7, the isomeric mixture of Formula 8 can be charged into a vessel with azobisisobutyronitrile and a solvent, such as heptane or anhydrous toluene, followed by thiophenol to cause isomerization to occur. The resulting reaction mixture is heated and stirred until the isomerization is complete. The solid compound of Formula 9 is collected, such as by vacuum filtration, and the isomerization is preferably complete to provide a high E/Z ratio, such as about at least about 35:1, at least about 50:1, at least about 75:1, at least about 100:1, or at least about 125:1.

In some embodiments, it may be useful to include further reaction materials to improve the Stage 7 isomerization reaction. For example, an activating material may be used to activate the thiophenol. In particular, it has been discovered that the use of certain materials both promotes and accelerates the isomerization reaction. Non-limiting examples of activating materials that may be used include activating metals or complexes thereof, particularly transition metals or complexes thereof, and more particularly zinc or complexes thereof. Surprisingly, zinc in particular has been found to provide this activating effect even when used in only a relatively small amount, such as a catalytic amount.

The amount of activating material used in the isomerization reaction may vary depending upon the exact material used. In specific embodiments, such as where a metal (e.g., activated zinc dust) is used, the amount of the activating material may be up to about 0.5 equivalents (based on the amount of the Stage 6 reaction product that is used in the isomerization reaction). In further embodiments, up to about 0.4, up to about 0.3, up to about 0.2, up to about 0.1, up to about 0.08, up to about 0.06, up to about 0.04, up to about 0.02, or up to about 0.01 equivalents of activating material may be used. In specific embodiments, the amount of activating material comprises about 0.001 to about 0.1 equivalents, about 0.005 to about 0.05 equivalents, or about 0.008 to about 0.03 equivalents.

The reaction product undergoes base hydrolysis in Stage 8. The methyl ester of Formula 9 can be combined with a solvent, such as THF, and a suitable base, such as NaOH. The reaction mixture can be stirred while an alcohol (e.g., methanol) is added to provide a homogeneous solution that is purged with nitrogen and refluxed until completion of the hydrolysis of the methyl ester to form the carboxylic acid of Formula 10.

The carboxylic acid can be converted to a carbamate (Formula 11) in Stage 9 of Reaction Scheme I. Preferably, the carboxylic acid can be combined with a suitable solvent such as anhydrous toluene. The formed solution can be then combined with diphenylphosphoryl azide and triethylamine and stirred until consumption of the starting carboxylic acid. Methanol can be later added, and the solution can be refluxed. The carbamate of Formula 11 can be isolated from the reaction solution and used in Stage 10 to prepare huperzine A (Formula 12).

In Stage 10, the carbamate can be combined with a suitable solvent (e.g., chloroform, acetonitrile, or toluene) and a halogenated trimethylsilane and refluxed. Methanol can be added and the resultant solution is again refluxed and followed by solvent removal. The resulting residue can be isolated to provide crude huperzine A.

The methods of the present invention can particularly be combined with any variety of synthetic methods for preparing huperzine A. For example, the following documents all disclose one or more synthesis steps in the preparation of huperzine A and are incorporated herein by reference in their entirety: U.S. Pat. No. 4,929,731; U.S. Pat. No. 5,104,880; U.S. Pat. No. 5,106,979; U.S. Pat. No. 5,547,960; U.S. Pat. No. 5,663,344; U.S. Pat. No. 5,869,672; U.S. Pat. No. 6,271,379; Bai, D. L., et al. Current Medicinal Chemistry 2000, 7, 355-374; Kozikowski, A. P., et al., J. Org. Chem.; 1991, 56, 4636-4645; Yamada, F, et al., J. Am. Chem. Soc. 1991, 113, 4695-6; Kaneko, S., et al., Heterocycles 1997, 46, 27-31; Kaneko, S., et al., Tetrahedron 1998 5471-5484; Chassaing, C., et al., Tetrahedron Letters 1999, 8805-9; He, X. C., et al., Tetrahedron: Asymmetry 2001, 12, 3213-3216; Kozikowski, A. P., et al., J. Chem. Soc., Perkin Trans. I, 1996, 1287-1297; Qian, L., et al., Tetrahedron Letters 1989, 30, 2089-2890; Chen, W. P., et al., J. Pharmaceut. 1991, 22, 256; and Hayashi, T., et al., Bull Chem. Soc. Jpn. 1980, 53, 1138. Any single step described in the present application could be incorporated into another known method, such as those incorporated above, to provide an improved method for preparing huperzine A.

The methods of the invention are also useful in the synthesis of analogs and derivatives of huperzine A. For example, huperzine A can be prepared according to the present invention and then subjected to further synthesis steps to produce a desired analog or derivative. For example, U.S. Pat. No. RE 38,460, which is incorporated herein by reference in its entirety, describes novel huperzine A derivatives and methods of synthesizing the derivatives by starting from huperzine A. Huperzine A prepared according to the present invention can particularly be used to prepare derivatives, such as described in RE 38,460. Accordingly, the present methods are understood to expressly encompass methods of preparing analogs and derivatives of huperzine A by preparing huperzine A according to the methods described herein and using the huperzine A in a method to prepare the derivative or analog. Thus, the present invention encompasses methods of preparing any of the compounds disclosed in RE 38,460, as well as other derivatives and analogs of huperzine A.

### EXPERIMENTAL

The present invention is more fully illustrated by the following examples, which are set forth to illustrate certain embodiments the present invention and are not to be construed as limiting.

### Example 1

### Preparation of (−)-Huperzine A

1,4-Cyclohexanedione monoethylene ketal (250 g, 1.0 equivalent), methyl propiolate (300 ml, 2.1 equivalents), isopropyl alcohol (4,000 ml) and ammoniacal methanol (7-8N, 2,625 ml) were charged in to a pressure vessel and sealed. The reaction mixture was heated to 135-140° C. with constant stirring and maintained for 9 hours. The mass was allowed to cool to 20-25° C., and the solvent was distilled under vacuum at 40-45° C. until about 70-75% of the solvent was removed. The solution was cooled to 0-5° C., stirred for 2 hours, and filtered. The filtered solid was washed with portions of cold isopropyl alcohol (0-5° C.) and dried under vacuum to yield the pyridone of Formula (2), 1′,5′,7′,8′-tetrahydro-spiro[1,3-dioxolane-2,6′(2′H)-quinolin]-2′-one (150 g, 45% yield, HPLC purity >98%).

The pyridone of Formula (2) (300 g, 1.0 equivalent) was combined with dichloromethane (3000 ml), 1N sodium hydroxide solution (1600 ml, 1.1 equivalent), and benzyl triethyl ammonium chloride (165 g, 0.5 equivalent) and stirred at 20-25° C. for 15 minutes. Silver carbonate (399 g, 1.1 equivalent) was added followed by iodomethane (270.4 ml, 3.0 equivalent) at 20-25° C. and stirred for 5 hours at the same temperature. An in-process analysis by HPLC showed <0.1% starting material (pyridone). Work-up was carried out by layer separation followed by distillation of the DCM layer to yield the O-methylated compound of Formula (3) (300 g, 95% yield, HPLC purity of 94%).

The O-methylated product (320 g) was combined with water (2,880 ml) and phosphoric acid (88%, 1,280 ml) and stirred at 20-25° C. for complete dissolution. The solution was slowly heated to 75-80° C. and maintained at that temperature for 3 hours. HPLC analysis carried out at the end of the 3 hour heating indicated <1% of the starting material remained. The reaction mass was cooled to 5-10° C. and pH adjusted to 7.0-7.5 by adding 50% sodium hydroxide solution. The resulting solution was then extracted with ethyl acetate (3 times with 1,280 ml each time) and distilled to yield the ketone compound of Formula (4) as a brown solid (240 g, 93% yield, HPLC purity of 92%).

Sodium hydride (50%, 27.7 g, 1.2 equivalents) and dimethyl carbonate (1,275 ml) were heated to 85-90° C. under nitrogen atmosphere, and the ketone of Formula (4) (85 g, 1.0 equivalent) diluted with dimethyl carbonate (1,275 ml) was added drop wise over a period of 1.5 hours. After addition, the reaction mixture was maintained at the same temperature for approximately 30 minutes. A sample for HPLC showed <1% of the ketone starting material remained. Dimethyl carbonate was then distilled off completely under vacuum at 40-45° C., and the residue was cooled to 10-15° C. Chilled water was added and dissolved completely. The pH was adjusted to 2-3 by adding 5 N HCl (160 ml) and extraction was performed with ethyl acetate (1 time with 340 ml and 2 times with 170 ml). The solvent was distilled off completely to get the crude β-keto ester of Formula (5).

The crude ester was dissolved in 800 ml 5% ethyl acetate:hexane mixture by heating at 60-65° C. The resulting mixture was allowed to cool to ambient temperature (20-25° C.) and filtered through filter paper. The solvent was distilled off completely under vacuum at 40-45° C. The resulting residue was stirred with hexane for 30 minutes at 20-25° C. The product was then collected by filtration and bed washed with portions of hexane. The product was dried under vacuum (740-750 mm/Hg) at 25-30° C. for 2 hours to yield pure product (80.2 g, 71% yield, HPLC purity-98%).

A chiral ligand according to Formula (13) (2.13 g, 2 mol %), allyl palladiumchloride dimer (0.56 g, 1 mol %), and acetone (140 ml) were combined and stirred at 20-25° C. for 1 hour under a nitrogen atmosphere. To the mixture was added 2-methylene-1,3-propanediol diacetate (26.2 ml, 1.0 equivalent) and 35 ml of acetone and the new mixture was maintained at the same temperature for 1 hour. A mixture of the purified keto ester of Formula (5) (35 g, 1.0 equivalent), 1,1,3,3-tetramethyl guanidine (42 ml, 2.2 equivalents), and acetone (175 ml) was added to the above solution in lots over a period of 30 minutes at 20-25° C. The resulting mixture was then stirred at the same temperature for 1 hour under a nitrogen atmosphere. A sample for chiral HPLC indicated <1% starting material (keto ester) remained. Acetone was then distilled off under vacuum at 40-45° C. to obtain a crude material. The Crude material was passed through silica gel column and eluted with hexane and ethyl acetate mixtures to remove catalyst and ligand. The fractions containing product were collected and the solvent was distilled completely to yield pure product of the compound of Formula (6) (35 g, 82% yield, HPLC purity of 78%).

This crude product (35 g) was stirred with isopropyl alcohol (140 ml) at 20-25° C. for 30 minutes. The obtained solid was filtered and washed with isopropyl alcohol (17.5 ml), and the material was dried under vacuum for 2-3 hours at 35-40° C. to get pure product as a white solid (21 g, 50% yield, HPLC purity of 97.5%).

To a mixture of the purified compound of Formula (6) (21 g, HPLC purity of 97.5%) and ethylene dichloride (210 ml) was added trifluoromethanesulphonic acid (21 ml) drop wise at 20-25° C., and the solution was stirred for 1 hour at the same temperature. An in-process analysis by HPLC showed <0.1% starting material (Formula (6) compound). The reaction mass was then cooled to 10-15° C. and neutralized with 10% sodium bicarbonate solution (315 ml). The layers were separated and the aqueous layer was extracted with ethylene dichloride (65 ml). The organic layer was dried over anhydrous sodium sulphate and distilled under vacuum at 40-45° C. to yield crude olefinic ester according to Formula (7). The crude material was purified by recrystallization using a mixture of heptanes as the recrystallization solvent.

The crude material was stirred with the heptanes (525 ml) at 90-95° C. for 30 minutes and filtered through filter paper at 80-85° C. After attaining 20-25° C., the solution was allowed to rest for 2 hours without agitation. The supernatant liquid was decanted, and the crystals formed at the bottom were isolated by stirring with heptanes (42 ml) followed by filtration. The filtered solid was then washed with portions of heptanes (21 ml) and dried under vacuum at 35-40° C. for 1 hour to yield pure product of the compound according to Formula (7) (16 g, 76% yield, HPLC purity of >99%). The yield was 37.5% from the corresponding β-ketoester.

Into a 2-L round-bottomed flask was charged ethyltriphenylphosphonium bromide (248 g, 668 mmol), followed by anhydrous tetrahydrofuran (1.0 L). The heterogeneous mixture was stirred while n-butyllithium (233 mL, 583 mmol, 2.5 M in hexane) was added over approximately 20 minutes. In an alternate embodiment, the n-butyllithium can be replaced with hexyllithium (e.g., 1.5 to 2.0 equivalents). The temperature was maintained at <30° C. with a water bath. The resulting reaction mixture was stirred at room temperature for 35 minutes then chilled to 0-2° C. with an ice-water bath. This was followed by the addition of the ester a compound of Formula (7) (48.0 g, 167 mmol) in THF (150 mL) over approximately 30 minutes. The temperature was maintained at 0-2° C. during the addition of the ester. After being stirred at 0-2° C. for 35 minutes the reaction mixture was warmed to room temperature and agitated until the completion of the reaction was detected by TLC (approximately 1 hour). The reaction was quenched with DI water (500 mL). The resulting heterogeneous mixture was concentrated under reduced pressure (>29 inches Hg, 40° C.). The milky residue was extracted with EtOAc (3 times with 300 mL). The combined organic phase was washed with 5% NaCl aqueous solution (3 times with 150 mL) then concentrated to give a semisolid mass (170 g). The residue was dissolved in methylene chloride (150 mL, technical grade) and loaded to a silica gel column (550 g). The column was eluted with heptanes-EtOAc (5:1 v/v) and fractions were collected. Fractions 2 and 3 were combined, concentrated, and thoroughly dried under high vacuum to give 49.8 g (99.6% yield) desired Wittig product (the Formula (8) compound) as a 3:1 mixture of Z- to E-isomers.

Into a 1-L round-bottomed flask at room temperature was charged the Wittig product (53.0 g, 177.0 mmol), followed by AIBN (20.0 g, 121.8 mmol) and anhydrous toluene (530 mL). After purging the vessel with nitrogen, thiophenol (29.2 g, 265.5 mmol) was injected. The resulting reaction mixture was heated to 85-87° C. and stirred at this temperature until the isomerization was completed by HPLC (about 22 hours). The reaction mixture was then cooled to room temperature and the solvent was removed under reduced pressure (>29 inches Hg, 50° C.). The oily residue was dissolved in heptanes (1.0 L) with heating, then cooled to room temperature and further chilled to 0-2° C. for 1 hour. The solid was collected by vacuum filtration (Whatman paper #3, Buchner funnel) and rinsed with cold heptanes (100 mL). The isolated product was dried under vacuum to give 34 g (64% yield) desired olefin (the Formula (9) compound) as a white powder having an E:Z ratio by HPLC of about 100:1.

Into a 2-L round-bottomed flask was charged the Formula (9) methyl ester (57.8 g, 193.1 mmol), followed by THF (360 mL) and 20% NaOH (80.0 g NaOH dissolved in 320 mL DI water). The reaction mixture was stirred at room temperature while methanol (500 mL) was added until a homogeneous solution was obtained. After being purged with nitrogen the reaction mixture was refluxed until the completion of the hydrolysis of methyl ester was noticed by HPLC (approximately 27 hours). The reaction mixture was cooled to room temperature. Volatiles were removed under reduced pressure. After neutralizing the aqueous residue with conc. hydrochloric acid to approximately pH 7, the product was extracted with ethyl acetate (4 times with 250 mL). The combined organic phase was washed with DI water (250 mL) and concentrated to a thick oil. The residue was dissolved in CH₂Cl₂ (100 mL) and loaded to a silica gel pad (160 g) in a sintered glass funnel. The pad was eluted first with heptanes-EtOAc (500 ml, 10/1 v/v) to remove the unreacted methyl esters, followed by eluting with EtOAc (800 mL). The ethyl acetate fraction was concentrated and dried under vacuum to give 52.0 g (94.5% yield) desired carboxylic acid (Formula (10) compound) as a very thick oil.

Into a 1-L round-bottomed flask was charged the Formula (10) carboxylic acid (20.0 g, 70.1 mmol), followed by anhydrous toluene (300 mL). The mixture was stirred to achieve a clear solution, followed by the additions of diphenylphosphoryl azide (19.3 g, 70.1 mmol) and triethylamine (7.24 g, 71.5 mmol). The resulting reaction mixture was stirred at 83° C. until the starting carboxylic acid was completely consumed (HPLC, approximately 2 hours). After cooling the reaction mixture to <64° C., methanol (300 mL) was added. The resulting reaction mixture was refluxed again for 24 hours. After being cooled to room temperature the reaction mixture was concentrated under reduced pressure. The oily residue (65 g) was dissolved in CH₂Cl₂ (50 mL) and loaded to a silica gel column (400 g). The column was eluted first with heptanes-EtOAc (1.5 L, 6/1, v/v), then with 3:1 heptanes-EtOAc until no more product could be detected by TLC. Fractions (˜200 mL each) containing pure product were combined, concentrated, and dried under vacuum to give 17.6 g (80% yield) of the desired carbamate of Formula (11) as a foamy solid.

Into a 100-mL round-bottomed flask at room temperature was charged the Formula (11) carbamate (1.0 g, 3.9 mmol), sodium iodide (2.9 g, 19.5 mmol), and acetonitrile (12 mL). After stirring at room temperature for 10 minutes, chlorotrimethylsilane (2.0 g, 18.3 mmol) was added and the resulting mixture was stirred at reflux for 4 hours. The reaction mixture was cooled to ambient temperature, diluted with dichloromethane (20 mL) and extracted with 1.5 M hydrochloric acid (13 mL). The aqueous acidic extract was washed twice with dichloromethane (2×18 ml) and then the pH adjusted to pH 9-10 with 6M sodium hydroxide (approximately 3.5 mL) and the mixture re-extracted three times with dichloromethane (3×12 mL). The latter three dichloromethane extracts were combined and washed with dibasic sodium phosphate solution (1.5 g in 15 mL water), dried, and concentrated to give 0.78 g (82% yield) crude (−)-huperzine A as an off-white powder. The crude (−)-huperzine A (0.78 g) was recrystallized from 1:1 acetonitrile/water (10 mL) to give 0.36 g (46% yield) of pure (−)-huperzine A as a white powder.

### Example 2

### Comparative of Reaction Times for Stage 3 Ketal Hydrolysis Using Phosphoric Acid Versus Other Acids

The O-methylated product (320 g) was combined with water (2,880 ml) and phosphoric acid (88%, 1,280 ml) and stirred at 20-25° C. for complete dissolution. The solution was slowly heated to 75-80° C. and maintained at that temperature for 3 hours. HPLC analysis carried out at the end of the 3 hour heating indicated <1% of the starting material remained. (HPLC purity of 92%).

Comparative reactions using 1N sulfuric acid or 1N hydrochloric acid were carried out at room temperature with stirring for 15 hours. In the reaction using sulfuric acid, only about 50% conversion of the starting material had been achieved after 15 hours. In the reaction using hydrochloric acid, HPLC analysis after 15 hours showed the product only had a purity of about 49%. Moreover, many impurities were also observed in this reaction product.

Many modifications and other embodiments of the inventions set forth herein will come to mind to one skilled in the art to which these inventions pertain having the benefit of the teachings presented in the foregoing descriptions. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A method of preparing huperzine A of a derivative thereof, the method comprising the following steps:
A) reacting the compound of Formula (1) with methyl propiolate to form the compound of Formula (2) 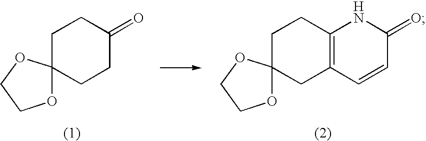
B) methylating the compound of Formula (2) to form the compound of Formula (3) 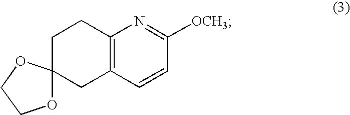
C) performing acid hydrolysis on the compound of Formula (3) using aqueous phosphoric acid to form the compound of Formula (4) 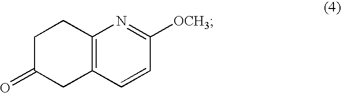
D) performing carboxymethylation on the compound of Formula (4) to form the compound of Formula (5) 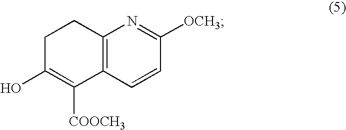
E) performing annulation on the compound of Formula (5) using acetone as the reaction solvent to form the compound of Formula (6) 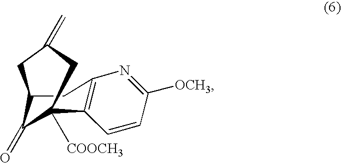
and performing isomerization on the compound of Formula (6) using ethylene dichloride as the reaction solvent to form the compound of Formula (7) 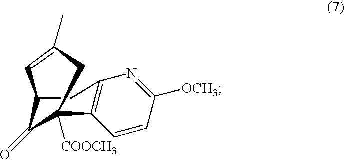
F) performing Wittig coupling on the compound of Formula (7) to form the compound of Formula (8) 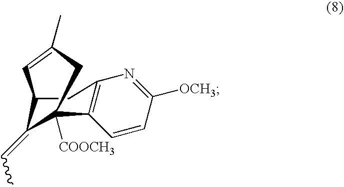
G) performing isomerization on the compound of Formula (8) to form the compound of Formula (9) 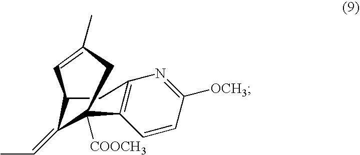
H) performing base hydrolysis on the compound of Formula (9) to form the compound of Formula (10) 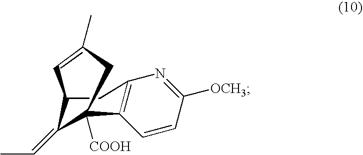
I) performing a Curtius rearrangement on the compound of Formula (10) to form the methyl ester of Formula (11) 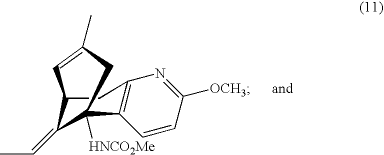
J) performing carbamate hydrolysis and cleavage of the methyl ester of Formula (11) to form huperzine A according to Formula (12)

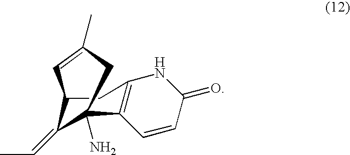

2. The method of claim 1, wherein the phosphoric acid of step C) has a concentration of about 1M to about 6M.

3. The method of claim 1, wherein the hydrolysis reaction of step C) is complete in a time of less than about 3 hours.

4. The method of claim 1, wherein the carboxymethylation step D) comprises contacting the compound of Formula (4) with sodium hydride.

5. The method of claim 4, further comprising using dimethyl carbonate in the reaction step.

6. The method of claim 5, wherein the dimethyl carbonate is present in an amount suitable to function as a reagent and a solvent.

7. The method of claim 4, wherein step D) is carried out in the express absence of tetrahydrofuran as a solvent.

8. The method of claim 4, wherein the compound of Formula (5) exhibits a purity, when measured by HPLC, of at least about 90%.

9. The method of claim 4, wherein the compound of Formula (5) exhibits a purity, when measured by HPLC, of at least about 97%.

10. The method of claim 1, wherein the annulation of step E) comprises a single recrystallization of the compound of Formula (6) using isopropyl alcohol as the recrystallization solvent.

11. The method of claim 10, wherein the recrystallized compound of Formula (6) exhibits an optical purity of at least about 95%.

12. The method of claim 1, wherein the isomerization of step E) is carried out at a temperature of less than 30° C., and wherein the isomerization is completed in a time of less than four hours.

13. The method of claim 1, wherein after the isomerization step G), the compound of Formula (9) has an E:Z ratio of at least about 50:1.

14. The method of claim 1, wherein said isomerization step G) comprises reacting the compound of Formula (8) with thiophenol activated with an activating material comprising a metal.

15. The method of claim 14, wherein the metal is zinc.

16. A method of preparing huperzine A or a derivative thereof, the method comprising the step of converting an intermediate compound of Formula (3) 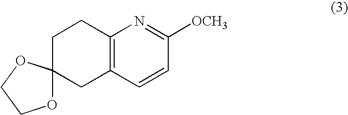
to an intermediate compound of Formula (4) 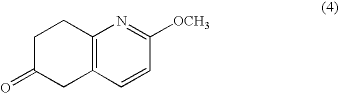
by acid hydrolysis, wherein the acid comprises aqueous phosphoric acid.

17. The method of claim 16, wherein the phosphoric acid has a concentration of about 1M to about 6M.

18. The method of claim 16, wherein the converting step is complete in a time of less than about 3 hours.

19. A method of preparing huperzine A or a derivative thereof, the method comprising the step of converting an intermediate compound of Formula (4) 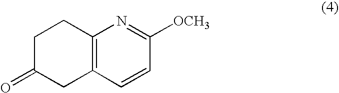
to an intermediate compound of Formula (5) 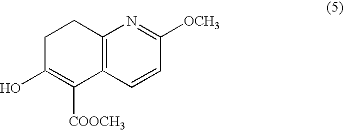
by contacting the compound of Formula (4) with sodium hydride in dimethyl carbonate, wherein the dimethyl carbonate is present in an amount suitable to function as a reagent and a solvent.

20. The method of claim 19, wherein the method step is carried out in the express absence of tetrahydrofuran as a solvent.

21. The method of claim 19, wherein the compound of Formula (5) exhibits a purity, when measured by HPLC, of at least about 90%.

22. The method of claim 19, wherein the compound of Formula (5) exhibits a purity, when measured by HPLC, of at least about 97%.

23. The method of claim 19, further comprising recrystallizing the compound of Formula (5) using hexane and ethyl acetate.

24. A method of preparing huperzine A or a derivative thereof, the method comprising converting a compound of Formula (5) 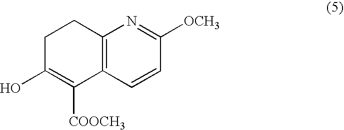
into a compound of Formula (6) 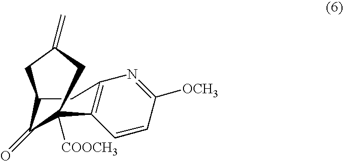
by performing an anulization reaction using acetone as the reaction solvent and converting the compound of Formula (6) into a compound of Formula (7) 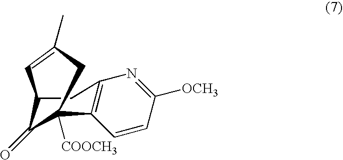
by performing an isomerization reaction using ethylene dichloride as the reaction solvent.

25. The method of claim 24, wherein the annulation further comprises a single recrystallization of the compound of Formula (6) using isopropyl alcohol as the recrystallization solvent.

26. The method of claim 25, wherein the recrystallized compound of Formula (6) exhibits an optical purity of at least about 95%.

27. The method of claim 25, wherein the isomerization reaction is carried out at a temperature of less than 30° C. and wherein the isomerization reaction is completed in a time of less than four hours.

28. A method of preparing huperzine A or a derivative thereof, the method comprising converting a compound of Formula (8) 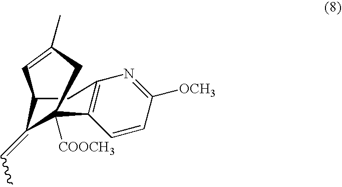
into a compound of Formula (9) 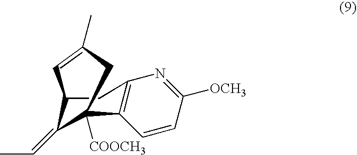
by performing isomerization on the compound of Formula (8) via a reaction with thiophenol activated with an activating material.

29. The method of claim 28, wherein the activating material comprises a metal.

30. The method of claim 29, wherein the metal is zinc.
